(19) United States
(12) Patent Application Publication
(10) Pub. No.: US 2009/0286718 A1
(43) Pub. Date: Nov. 19, 2009

(54) Stable Aqueous Cyclosporin Compositions

(75) Inventor: William Francis STRINGER, St. Petersburg, FL (US)

Correspondence Address: BRINKS, HOFER, GILSON & LIONE, P.O. BOX 1340, MORRISVILLE, NC, 27560, US

(73) Assignee: SIRION THERAPEUTICS, INC., Tampa, FL (US)

(21) Appl. No.: 12/272,445

(22) Filed: Nov. 17, 2008

Related U.S. Application Data
(60) Provisional application No. 61/019,088, filed on Jan. 4, 2008.

(51) Int. Cl. A61K 38/13; A61P 27/02

(52) U.S. Cl. 514/11

(57) ABSTRACT

An aqueous ophthalmic composition is disclosed. The composition may comprise a cyclosporin in an amount from about 0.001 to about 1%, glycerin, and purified water, wherein the composition is substantially free of NaCl and sodium bisulfite or sodium metabisulfite. The composition is useful for the treatment of ocular conditions.

### RELATED APPLICATIONS

The present patent document claims the benefit of the filing date under 35 U.S.C. §119(e) of Provisional U.S. Patent Application Ser. No. 61/019,088, filed Jan. 4, 2008, which is hereby incorporated by reference.

### FIELD

The present invention relates to ophthalmic pharmaceutical compositions comprising aqueous solutions of cyclosporin for the treatment of different ocular conditions using the ophthalmic pharmaceutical compositions.

### BACKGROUND

Cyclosporins are a group of nonpolar cyclic oligopeptides with immunosuppressant, anti-inflammatory, and anti-parasitic properties. Cyclosporin-A (CsA) has been used as an immune suppressor in application such as psoriasis, lymphoma, myelodysplastic syndrome, Sjogren's syndrome, corneal transplantation, and dry eye syndrome. In humans, CsA has been used as a topical formulation at concentrations ranging from 2% to lower concentrations such as about 0.01% to about 0.05%.

It is generally believed that the topical application of CsA to the eye significantly reduces the number of active lymphocytes in the conjunctiva and lacrimal glands. Thus, because CsA stimulates the secretion of tears by the principal lacrimal gland and accessory lacrimal glands, and avoids acinar cell apoptosis induced by lymphocytes, it may provide treatment for dry eye syndrome.

However, utility and effectiveness of cyclosporins, such as cyclosporin A, in treating diseases and conditions of the eye has been limited by the lack of compositions that are acceptable to the eye, for example, as eye-drops. For effective patient compliance, eye-drops of cyclosporins providing minimal patient discomfort and a convenient administration regimen are required.

However, the insolubility of cyclosporins in water is an ongoing problem in the formulation of these compounds. This often leads to precipitation of the cyclosporin from aqueous-based eye-drops resulting in strong irritation of the eye. The stability of cyclosporins in aqueous-based eye-drops is also important to provide an adequate shelf life of these compounds at room temperature.

Efforts have been made to overcome these difficulties by dissolving cyclosporin in oils, such as vegetable oils. In oil-containing solutions, however, cyclosporin is typically poorly distributed in the eyes, thus requiring high concentrations (≧2%) of cyclosporin for effective clinical treatment. Further, these oil-containing solutions typically cause a disagreeable feeling to the eyes, which leads to poor patient compliance issues. [there are also published reports of oil damaging the corneal surface over long periods of time]

Other efforts to overcome the insolubility of cyclosporins in oils have resulted in oil in water emulsions, but as a practical matter, these have resulted in only low, effective doses of cyclosporin formulations.

In an attempt to solve these problems, studies conducted with various surfactants, which are currently used for formulating medical substances with low solubility in water, for example, polyoxyethylene 20 sorbitan monooleate (Polysorbate 80) and polyoxyethylene hydrogenated castor oil, have been disclosed (for example, in U.S. Pat. No. 5,951,971).

Co-assigned Mexican PCT application WO 2004/096261, discloses that the ophthalmic solution Sophisen®, (as disclosed in U.S. Pat. No. 6,071,958), allows the solubilization of cyclosporin-A. The disclosed solutions contain surface-active, emulsifying, antibacterial, and antioxidant components, such as sodium bisulfate, sodium metametabisulfite, and ionic tonicity agents. The latter two ingredients, in combination with emulsifying agents, at neutral or acidic pH levels, typically result in stinging and burning of the eyes. Further, these solutions may not be optimized for the antibacterial preservative employed therein.

### SUMMARY

The present applicant unexpectedly has discovered that some or all of the aforementioned difficulties may be overcome with a composition comprising cyclosporin, glycerin, and water where the composition contains less than about 0.3% sodium chloride and less than about 0.04% sodium bisulfite or sodium metabisulfite, as disclosed herein.

In one aspect, the composition includes a cyclosporin in an amount of from about 0.001% to about 1%, glycerin in an amount between about 0.1% and 5%, and purified water. The composition also contains less than about 0.3% sodium chloride and less than about 0.04% sodium bisulfite or sodium metabisulfite.

In a feature of this aspect, the composition is substantially free of sodium chloride and sodium bisulfite or sodium metabisulfite. The composition may further comprise a polyoxyethylene sorbitan fatty acid ester and a polyoxyethylene fatty acid ester in a total amount between 3% and 8%.

In another aspect, an aqueous ophthalmic composition comprises cyclosporin in an amount from about 0.001% to about 0.5%, a polyoxyethylene sorbitan fatty acid ester and a polyoxyethylene alkyl ether in a total amount between 3% and 8%, glycerin in an amount from about 0.1% to about 5%, ethanol in an amount from about 0.2% to about 0.5%, sorbic acid in an amount from about 0.1% to about 0.5%, and purified water. The pH of the composition may be between 6.0 and 7.5, and the composition contains less than about 0.3% sodium chloride and less than about 0.04% sodium bisulfite or sodium metabisulfite.

Additionally, a method of treating an ocular condition is disclosed. The method includes contacting ocular tissue with an aqueous composition comprising a cyclosporin in an amount of from about 0.001% to about 1%, glycerin in an amount between about 0.1% and about 5%, and purified water. The composition contains less than about 0.3% sodium chloride and less than about 0.04% sodium bisulfite or sodium metabisulfite.

### DETAILED DESCRIPTION

An aqueous ophthalmic composition having improved stability and that can provide increased comfort is disclosed. The composition includes a cyclosporin in an amount of from about 0.001% to about 1%, glycerin in an amount between about 0.1% and about 5%, and purified water. The composition contains less than about 0.3% sodium chloride and less than about 0.04% sodium bisulfite or sodium metabisulfite. Preferably, the composition is in a pH range between about 6.0 to 7.5

As used herein, unless otherwise specified, the concentration of a component or ingredient of a composition is represented by mass of the component or ingredient per total volume of the composition (i.e., g/mL), and is typically expressed as a percentage. For example, a concentration of 1% means 1 g per 100 mL of the composition.

The terms “cyclosporin” and “ciclosporin” are used interchangeably herein and include naturally occurring fungal metabolites, such as the cyclosporin A, B, C, D and G, as well as synthetic and semi-synthetic cyclosporins, for example the dihydro- and iso-cyclosporins, [(D)-Ser]8-Ciclosporin, [0-acetyl,(D)-Ser]8-Ciclosporin, [beta-fluoro-(D)Ala]8-Ciclosporin, [Val]2-[(D)methylthio-Sar]3- and [Dihydro-MeBmt]1-[Val]2-[(D)methylthio-Sar]3-Ciclosporin, [0-(2-hydroxyethyl)-(D)Ser]8-Ciclosporin, and [3′-deshydroxy-3′-keto-MeBmt]1-[Val]2-Ciclosporin. The preferred cyclosporin is cyclosporin A (CsA). Mixtures of at least two different cyclosporins may be used. The cyclosporin is advantageously administered topically as an aqueous, non-oil-in-water emulsified ophthalmic drop containing an effective amount of the cyclosporin. Concentrations of about 0.01 to 1%, preferably about 0.05 to 0.5%, of a cyclosporin may be used. The cyclosporin may be administered topically in any quantity required to provide amelioration or elimination of an ocular condition. For example, 5 microliters to 1 milliliter of a solution containing an effective amount of a cyclosporin, such as about 0.01 to 1%, preferably about 0.05 to about 0.5%, of cyclosporin is useful.

As mentioned previously, a difficulty in using cyclosporin as an active ingredient in treatment of ocular tissue has been finding a way to deliver cyclosporin to the ocular tissue without irritation. While the known ophthalmic solution Sophisen® has been used to deliver cyclosporin to the eye, the formulation has the drawbacks that it is irritating to the eye and has less than optimal stability for some of the components used therein. The presently disclosed composition provides an improvement over previously known ophthalmic compositions in that it provides improved component stability and can provide increased comfort. Specifically, the antimicrobial preservative effectiveness of the present composition is improved. Sorbic acid has been found to be an effective antimicrobial agent for ophthalmic solutions at certain pH values. It would have been expected that the effectiveness of sorbic acid would be optimal at the pKa of sorbic acid, i.e., 4.67. Surprisingly, in the present solution, a pH of 6.0 to 6.5 is optimal for sorbic acid stability while still providing antimicrobial effectiveness. Further, it is believed that the present solution will provide improved comfort, while also providing the above-mentioned improved stability. Specifically, glycerin in its correct proportion provides tonicity while not detrimentally affecting stability. Further improvements include removing most or substantially all, if not all, sodium chloride, sodium bisulfite, and sodium metabisulfite while maintaining stability. In sum, it would not have been expected that moving the solution pH away from the pKa of sorbic acid would provide stability for the sorbic acid while maintaining the antimicrobial effectiveness of the sorbic acid in the present aqueous ophthalmic solution. In addition to the improved antimicrobial effectiveness and stability, it is unexpected that such a solution could potentially provide improved comfort as well. All of the above features combine to provide a uniquely stable aqueous ophthalmic solution.

As used herein, the term “ocular comfort” refers to an effect of an ophthalmic composition on a user upon contact of the composition with an ocular tissue of the subject. Ocular comfort may be determined by a subject responding to the introduction of drops of a composition into the eye of the subject. By way of example, the response may be graded on a numerical scale, from 1 to 10, 1 representing mostly discomfort, and 10 representing mostly comfort or the response may be an indication that the ocular comfort is acceptable or unacceptable. Additionally, ocular comfort may be determined by appropriate studies in animals, such as rabbits, where the lack of irritation may be determined by observation of the animal. Preferably, the ophthalmic composition disclosed herein has a graded value at least one higher than that of an ophthalmic composition comprising higher amounts of sodium metabisulfite, sodium bisulfate, and/or sodium chloride. More preferably, the value is at least two higher.

Additionally, as used herein, “ocular tissue” refers to any tissue adjacent or in communication with the eye. For example, ocular tissue includes eyelids, sclera, cornea, eyeball and any of the aforementioned supporting structures/tissues.

One way in which ocular comfort can be improved in the instant composition is by using non-ionic tonicity agents that would be less irritating to the eye than sodium chloride (NaCl). Sodium chloride is a known tonicity agent and is traditionally used in ophthalmic pharmaceutical formulations to make the formulation isotonic to tears. The ophthalmic compositions disclosed herein may be adjusted with non-ionic tonicity agents to approximate the osmotic pressure of normal lachrymal fluids, which, as stated in U.S. Pat. No. 6,274,626, is equivalent to a 2.5% solution of glycerin. Osmotic pressure, measured as osmolality, is generally about 225 to 400 mOsm/kg for conventional ophthalmic solutions. In the present composition, suitable non-ionic tonicity adjustment agents may include, but are not limited to, glycerin, and polyalcohols such as glucose, sorbitol, mannitol, polyethylene glycol and propylene glycol. Preferred tonicity adjustment agents include glycerin and propylene glycol. The ophthalmic compositions disclosed herein are substantially free of ionic tonicity agents such as sodium chloride or potassium chloride. More preferred is glycerin as the non-ionic tonicity agent in a concentration of from about 0.1% to about 5%, preferably from about 1% to about 3%, more preferably about 1.15% such that the composition has an osmolality from about 200 to about 700 mOsm/kg, preferably from about 200 to about 400 mOsm/kg.

As used herein, the phrase “free or substantially free” refers to a composition essentially absent of a particular chemical or compound, or a composition where the amount of particular chemical or compound is less than the amount needed to cause ocular discomfort or cause stabilization of the composition. For example, “substantially free of sodium chloride” refers to a composition containing less than about 0.2% sodium chloride. Preferably, “substantially free of sodium chloride” refers to a composition containing less than about 0.03% sodium chloride. More preferably, “substantially free of sodium chloride” refers to a composition containing less than about 0.003% sodium chloride. Most preferably sodium chloride is absent from the composition.

To improve comfort in the present composition, the composition is substantially free of sodium bisulfite or sodium metabisulfite. Sodium bisulfite or sodium metabisulfite, which are known oxygen scavengers, may be used in pharmaceutical formulations as stabilizing agents. The applicant has unexpectedly found that an aqueous ophthalmic composition comprising cyclosporin, glycerin, and water and containing less than about 0.04% sodium bisulfite or sodium metabisulfite, as described herein, is stable despite being substantially free of sodium bisulfite or sodium metabisulfite. Advantageously, the composition is believed to be more comfortable when sodium bisulfite or sodium metabisulfite is at a low concentration or the solution is substantially free of sodium metabisulfite or sodium bisulfite.

As with sodium chloride, the phrase “substantially free of sodium bisulfite or sodium metabisulfite” refers to a composition containing less than about 0.04% sodium bisulfite or sodium metabisulfite. Preferably, “substantially free of sodium bisulfite or sodium metabisulfite” refers to a composition containing less than about 0.004% sodium bisulfite or sodium metabisulfite. More preferably, “substantially free of sodium bisulfite or sodium metabisulfite” refers to a composition containing less than about 0.0004% sodium bisulfite or sodium metabisulfite. Most preferably sodium bisulfite or sodium metabisulfite is absent from the composition.

The composition further comprises a surfactant that may be comfortably used in treatment of ocular tissue. The surfactant may comprise polyoxyethylene sorbitan fatty acid esters, polyoxyethylene fatty acid esters, polyoxyethylene alkyl ethers, polyoxyethylene castor oil derivatives, or combinations thereof. As used herein, polyoxyethylene sorbitan fatty acid esters are based on fatty acid esters of sorbitol copolymerized with ethylene oxide. An example is polyoxyethylene 20 sorbitan monooleate (Polysorbate 80), which has a hydrophilic-lipophilic balance (HLB) value of about 15, an acid value of about 2, a hydroxyl value of about 65-80, and a saponification value of about 45-55. The weight ratio of the polyoxyethylene 20 sorbitan monooleate (Polysorbate 80) to cyclosporin in the aqueous ophthalmic composition may be from about 1:1 to about 10:1. Preferably, the weight ratio of the polyoxyethylene 20 sorbitan monooleate (Polysorbate 80) to cyclosporin is from about 4:1 to about 7:1.

As used herein, polyoxyethylene fatty acid esters are based on saturated fatty acids, preferably not containing any substituent, having a chain length from 14 to 22 carbon atoms, preferably, 16 to 18 carbon atoms. Exemplary polyoxyethylene fatty acid esters include polyoxyethylene stearate. Preferably the polyoxyethylene stearate ester is a monoester. Preferably the polymerization number of the polyoxyethylene moiety is from about 20 to about 60. An example is polyoxyethylene 40 monostearate (polyoxyl 40 stearate), which has a HLB value of about 16.9, an acid value of less than 1, a hydroxyl value of about 27-40, and a saponification value of about 25-35. The weight ratio of the polyoxyethylene 40 monstearate (polyoxyl 40 stearate) to cyclosporin in the aqueous ophthalmic composition may be from about 25:1 to about 100:1. Preferably, the weight ratio of the polyoxyethylene 40 monstearate (polyoxyl 40 stearate) to cyclosporin is from about 50:1 to about 75:1.

As used herein, polyoxyethylene alkyl ethers are based on fatty alcohols having, for example, the structural formula CH₃(CH₂)ₓ(OCH₂CH₂)_{y}OH, where x is from about 12-18 and y is about 10-60. An example is a polyoxyl lauryl ether, which has a HLB value of about 16.9, an acid value of less than 5, a hydroxyl value of about 40 to 60 and a density of about 1.05. The weight ratio of the polyoxyethylene alkyl ether to cyclosporin may be from about 25:1 to about 100:1. Preferably, the weight ratio of the polyoxyethylene alkyl ether to cyclosporin is from about 40:1 to about 75:1. A preferred example of a polyoxyl lauryl ether is Brij 35 (also known as laureth-23).

When used in combination in one embodiment, the total amount of the polyoxyethylene sorbitan fatty acid ester and the polyoxyethylene fatty acid ester present in the aqueous ophthalmic composition may be between about 3% and about 8%. A preferred total amount of the polyoxyethylene sorbitan fatty acid ester and the polyoxyethylene fatty acid ester present in the aqueous ophthalmic composition is between about 4% and about 8%, more preferably between about 5% and about 8%. For example, the concentration of the polyoxyethylene sorbitan fatty acid ester and the polyoxyethylene fatty acid ester present in the aqueous ophthalmic composition may be between about 0.50% and about 0.55% and about 7%, respectively.

When used in combination in another embodiment, the total concentration of the polyoxyethylene sorbitan fatty acid ester and the polyoxyethylene alkyl ether present in the aqueous ophthalmic composition may be between about 5% and about 8%. For example, the concentration of the polyoxyethylene sorbitan fatty acid ester and the polyoxyethylene alkyl ether present in the aqueous ophthalmic composition may be between about 0.50 and about 0.55% and about 5%, respectively. The concentration of the polyoxyethylene sorbitan fatty acid ester and the polyoxyethylene alkyl ether present in the aqueous ophthalmic composition may be between about 0.50 and about 0.55% and about 7%, respectively.

When polyoxyethylene fatty acid esters or polyoxyethylene alkyl ethers are used in ophthalmic compositions in an amount greater than about 3%, together with ionic tonicity agents and/or sodium bisulfite or sodium-metabisulfite, with pH values of about 7, stinging and irritation may result. When the aqueous ophthalmic composition comprising cyclosporin, glycerin, and water containing less than about 0.3% sodium chloride and less than about 0.04% sodium bisulfite or sodium metabisulfite is used with a polyoxyethylene fatty acid ester or a polyoxyethylene alkyl ether in amounts of 4% or more, respectively, it is unexpectedly found that the ophthalmic composition has acceptable ocular comfort and extended stability. Preferably, the pH of the aqueous ophthalmic composition comprising cyclosporin, glycerin, and water containing less than about 0.3% sodium chloride and less than about 0.04% sodium bisulfite or sodium metabisulfite, with a polyoxyethylene fatty acid ester or a polyoxyethylene alkyl ether in amounts of about 4% or more is between about 6.0 and about 7.5. More preferably, the pH is about 6.5.

The ophthalmic composition may also contain a suitable antimicrobial preservative agent such as sorbic acid, benzalkoniam chloride, polyhexanide, and/or quaternary ammonium compounds. Antimicrobial preservatives are frequently used in ophthalmic preparations. In fact, these preservatives are required in multidose ophthalmic preparations in order to minimize contamination and infection by the end user. The antimicrobial preservative should be stable, i.e., not degrade, over the shelf life of the product. The ophthalmic composition may include sorbic acid in an amount of about 0.1 to about 0.5%. When sorbic acid is used as the antimicrobial preservative agent, the pH may be adjusted to about 6.5.

If an antimicrobial preservative agent is present in the ophthalmic composition, it preferably possesses suitable antimicrobial effectiveness as measured by established means, e.g., USP antimicrobial effectiveness tests. It is conventionally believed that the antimicrobial effectiveness of sorbic acid is enhanced if the aqueous composition has a pH close to the pKa of sorbic acid (4.67).

The applicant has found that sorbic acid degradation compromises the effectiveness of sorbic acid in aqueous ophthalmic compositions comprising cyclosporin, glycerin, sorbic acid, and water containing less than about 0.3% sodium chloride and less than about 0.04% sodium bisulfite or sodium metabisulfite, as described herein, at pH's less than 6.0. Unexpectedly, at a pH between 6.0 and 7.5, which is moving away from sorbic acid's pKa, the sorbic acid concentration provides antimicrobial effectiveness in a stable solution. It is important for the sorbic acid to remain stable in order to effectively function as an antimicrobial agent. The more stable the antimicrobial agent, the longer shelf life the composition will have. With regard to ocular comfort, a composition with a pH in the range of 6.0 to 7.5 can be more comfortable to ocular tissue than a composition with a lower pH. Thus, advantageously, the present composition in the preferred pH range is more stable and can provide greater ocular comfort.

As used herein, the term “degradation” refers generally to an active agent or a preservative that has changed chemically such that a pharmaceutical or pharmacological property of the active agent or preservative is reduced or eliminated. Alternatively, a physical property such as solubility, stability, or physical appearance is changed. Methods of determining the amount of degradation of active agents or preservatives and concentrations of initial active agent or preservatives remaining after an interval of time has elapsed are generally known. For example, an active agent that is detectable by a detection method generally used to determine a concentration of the active agent may be used to determine whether the concentration of the active agent has decreased relative to its initial formulated concentration. The detection method may only measure the concentration of active ingredient or may characterize any other component of the composition for the purpose of measuring degradation, such as a known degradation product. Visual inspection of the physical appearance of a solution of the composition may also provide a qualitative indication of stability.

The ophthalmic compositions disclosed herein may further include metal chelators. For example the ophthalmic compositions may include ethylene diamine tetraacetic acid (EDTA) in an amount from about 0.01% to about 1%.

With further regard to pH, typically, only small amounts of an acid or base will be needed to adjust the initial pH of the solution. By way of example, acids and bases suitable for adjusting the pH are hydrochloric acid, sodium hydroxide, fumaric acid and fumaric acid/sodium fumarate. The ophthalmic compositions comprising cyclosporin, a nonionic tonicity agent such as glycerin, and water, may optionally include a buffer system to maintain the pH of the composition. Preferably, the solution pH is adjusted without using both an acid and a base to avoid the formation of salts. The ophthalmic composition may include boric acid in an amount from about 0.01% to about 0.2%, and/or sodium borate in an amount from about 0.01 to about 0.5%. Additional ranges of boric acid and/or sodium borate may be used.

The ophthalmic compositions typically have a pH from 4 to 7.5, preferably from about 6.0 to about 7.0, most preferably about 6.5. A buffer (e.g., buffers including citrates, phosphates, borates, bicarbonates, etc.; or a buffer with intrinsic antimicrobial properties such as a sodium borate/boric acid buffer) may also be used to achieve (and maintain) the desired pH of the compositions.

The ophthalmic composition may also contain an antihistamine and/or mast cell stabilizer. For example, the antihistamine/mast cell stabilizer may be ketotifen, norketotifen, 10-hydroxy-detotifen or 10-hydroxy-norketotifen, or ophthalmically acceptable salts and/or optical isomers of these compounds. The antihistamine and/or mast cell stabilizer may be present in the composition in any effective concentration. Preferably, the concentration is about 0.01% to about 0.5%, more preferably about 0.02% to about 0.4%, most preferably about 0.03% to about 0.15%.

The ophthalmic composition may also contain a steroidal anti-inflammatory agent. Preferred steroidal anti-inflammatory agents are the corticosteroids. Preferred corticosteroids include alclometasone, amcinonide, betamethasone, betamethasone, betamethasone valerate, clobetasol, clocortolone, cortisol, cortisone, desonide, desoximetasone, dexamethasone, diflorasone, difluprednate, flumethasone, fluocinolone acetonide, fluocinonide, fluorometholone, fluprednisolone, flurandrenolide, flurandrenolone acetonide, fluticasone, halcinonide, halobetasol, hydrocortisone, methylprednisolone, mometasone, prednicarbate, prednisolone, prednisone, triamcinolone, and mixtures thereof.

The steroidal anti-inflammatory agent may be present in the composition in any effective concentration. Preferably, the concentration is about 0.01% to about 5%, preferably about 0.02% to about 3%, more preferably about 0.1% to about 2%.

The ophthalmic composition may also contain a non-steroidal anti-inflammatory drug (NSAID) suitable for topical application to ocular tissue. For example, the NSAID may include bromfenac (Xibrom), ketorolac (Acular), diclofenac (Voltaren), or flurbiprofen (Ocufen). The non-steroidal anti-inflammatory drug (NSAID) may be present in the composition in any effective concentration. Preferably, the concentration is about 0.01% to about 5%, preferably about 0.02% to about 3%, more preferably about 0.1% to about 2%.

The compositions disclosed herein comprising cyclosporin, glycerin, and water, where the composition is free or substantially free of sodium chloride and/or sodium bisulfite or sodium metabisulfite are unexpectedly stable. In such compositions, no more than about 10% of the cyclosporin and no more than about 20% of the sorbic acid are degraded at 55° C. and 40% RH for at least four weeks. The stabilization of the ophthalmic composition of cyclosporin may be such that no more than about 10% of the cyclosporin is degraded at 25° C. and 40% RH for at least four weeks. The above stabilities may extend for an even longer period of time, for example, two, three, four, five, six, or twelve months. The stability of the aqueous ophthalmic composition comprising cyclosporin, glycerin and water containing less than about 0.3% sodium chloride and less than about 0.04% sodium bisulfite or sodium metabisulfite, results in less than about 20% degradation of cyclosporin. Preferably, less than about 15%, more preferably less than about 10%, most preferably less than about 5% by weight of the cyclosporin is degraded.

The compositions disclosed herein may be free or substantially free of polymers comprising chitosan; linear polysaccharide compounds such as hyaluronic acid compounds; biocompatible polymers/thickeners such as polyoxyethylene-polyoxypropylene copolymers and acrylic acid homo- and co-polymers; and/or active agents other than cyclosporin.

Ophthalmic compositions as disclosed herein may also be useful for the treatment of dry eye condition, including inflammatory dry eye condition. Ophthalmic compositions may be formulated as single or multi dose units, with or without the use of a preservative, and may be manufactured by mixing various ingredients. The compositions may be packaged in single or multiple dosage forms, such as closed bottles, tubes, vials, or other containers made from materials such as glass or plastic.

The ophthalmic composition as disclosed herein is preferably essentially free of an oil-in-water emulsion. Further, the composition preferably is a topical composition. For example, the topical composition may be in the form of eye drops. The ophthalmic composition as disclosed herein may show significantly greater corneal penetration of cyclosporins than similar compositions that do not contain such a combination of compounds or are otherwise oil-in-water emulsions.

The ophthalmic compositions disclosed herein may be used for the treatment of ocular conditions. Ocular conditions include, for example dry eye disease, including inflammatory dry eye disease, allergies, allergic conjunctivitis, keratoconjunctivitis, pink eye, itchy eye, or combinations thereof. Methods of treating ocular conditions comprise administering to a human subject suffering from dry eye disease an effective amount of an ophthalmic composition described herein. The effective amount is any amount that would reduce or eliminate the etiology or the symptomology of the ocular condition. The compositions disclosed herein may be administered as drops, with one drop of the composition being applied to an eye of a subject suffering from or susceptible to allergic conjunctivitis two times per day, although more or less of the composition may be used in more or less frequent doses depending on multiple factors, including the makeup of the particular composition and the symptoms presented by the subject.

The ophthalmic compositions may be used, for example, for the treatment and temporary prevention of the signs and symptoms of allergic conjunctivitis, including itching of the eye and redness of the eye. Methods of treating allergic conjunctivitis comprise administering to a human subject suffering from or susceptible to allergic conjunctivitis an effective amount of an ophthalmic composition described herein.

The compositions disclosed herein may be used, for example, to treat, ameliorate, or reduce a condition resulting from dry eye and/or allergy. For example, a composition of the present invention can be applied topically to treat, ameliorate, or reduce the severity of, dry eye or symptoms thereof, allergic conjunctivitis or symptoms thereof, such as pink eye, itchy eye, or combinations thereof.

The ophthalmic compositions disclosed herein may be formulated as single or multi dose units, and may be manufactured by mixing the ingredients. The compositions may be packaged in single or multiple dosage forms, such as closed bottles, tubes, vials, or other containers made from materials such as glass or plastic.

### EXAMPLES

The following examples are illustrative of the embodiments of the present invention and are not to be interpreted as limiting or restrictive. Notwithstanding that the numerical ranges and parameters setting forth the broad scope of the invention are approximations, the numerical values set forth in the specific examples are reported as precisely as possible. Any numerical value, however, inherently contains certain uncertainties, as expressed by the standard deviation found in its respective measurements (e.g., pH), where such standard deviation can be determined or estimated. By way of example, a pH value is to be regarded as to be within a range of +/−0.2. The examples described below are represented in Tables 1 and 2.

### Example 1

### Control

To 170.04 g of water heated to 70° C. was added 14.01 g of Polyoxyethylene 40 Stearate and the resultant solution was allowed to cool to 55° C. To this solution 0.2032 g of EDTA dihydrate, 0.6008 g of sodium chloride, 0.1912 g of boric acid and 0.4404 g sorbic acid were added and stirred until dissolved. The solution was allowed to cool to room temperature and 0.0802 g sodium bisulfite or sodium metabisulfite was added. The resulting solution was designated Phase I Control.

To 0.7852 g of ethanol was added 0.2015 g of cyclosporin. The cyclosporin was mixed until completely dissolved. Polysorbate 80 (1.0755 g) was added to the solution and stirred until dissolved. The resulting solution was designated Phase II Control.

The Phase II Control solution was quantitatively added to the Phase I Control solution. Overnight mixing completely dissolved the cyclosporin. The resulting solution was designated Phase III Control Solution. The Phase III Control solution was diluted to a final weight of 200.03 g.

An alternative control solution was made essentially as described above for the Phase I Control solution with the following modification. The aqueous solution of Polyoxyethylene 40 Stearate, EDTA dihydrate, sodium chloride, boric acid and sorbic acid were held at a temperature of 55° C. for 30 minutes before cooling to room temperature. Then sodium bisulfite or sodium metabisulfite was added. Alternative Phase II and Phase III Control Solutions were prepared as above. The contents of each of these control solutions are represented in Table 1 collectively as Control.

### Sample A

To 163.09 g of water heated to 70° C. was added 14.04 g of Polyoxyethylene 40 Stearate and the solution was allowed to cool to 55° C. To this solution 0.2014 g of EDTA dihydrate, 2.33 g of glycerin, 0.1913 g of boric acid and 0.4413 g sorbic acid were added and stirred until dissolved. The solution was held at a temperature of 55° C. for 30 minutes and then allowed to cool to room temperature. This solution was designated Phase IA.

To 0.7933 g ethanol was added 0.2019 g of cyclosporin. The cyclosporin was mixed until completely dissolved. Polysorbate 80 (1.0741 g) was added to the solution and stirred until dissolved. This solution was designated Phase IIA.

The Phase II A solution was quantitatively added to the Phase IA solution. Overnight mixing completely dissolved the cyclosporin. This solution was diluted to a final weight of 200.02 g and was designated Sample A.

### Sample B

To 164.64 g of water heated to 70° C. and 13.99 g of Brij 35 was added and the solution was then allowed to cool to 55° C. To this solution 0.2001 g of EDTA dihydrate, 2.33 g of glycerin, 0.1911 g of boric acid and 0.4413 g sorbic acid were added and stirred until dissolved. The resulting solution was held at a temperature of 55° C. for 30 minutes and then allowed to cool to room temperature. This solution was designated Phase IB.

To 0.7975 g of ethanol was added 0.2002 g of cyclosporin with mixing until the cyclosporin completely dissolved. Polysorbate 80 (1.0789 g) was then added to the solution and stirred until dissolved. This solution was designated Phase IIB.

The Phase IIB solution was quantitatively added to Phase IB solution. Overnight mixing completely dissolved the cyclosporin. The solution was diluted to a final weight of 200.03 g and designated Sample B.

### Sample C

To 160.16 g of water heated to 70° C. was added 11.98 g of Brij 35 and the solution was then allowed to cool to 55° C. To this solution 0.1999 g of EDTA dihydrate, 2.33 g of glycerin, 0.1899 g of boric acid and 0.1112 g sorbic acid were added and stirred until dissolved. The solution was held at a temperature of 55° C. for 30 minutes then allowed to cool to room temperature. This solution was designated Phase IC.

To 0.7912 g of ethanol was added 0.2009 g of cyclosporin with mixing until the cyclosporin completely dissolved. Polysorbate 80 (1.0796 g) was then added to the solution and stirred until dissolved. This solution was designated Phase IIC.

The Phase IIC solution was quantitatively added to Phase IC solution. Overnight mixing completely dissolved the cyclosporin. The solution was diluted to a final weight of 200.02 g and designated Sample C.

### Sample D

To 163.62 g of water heated to 70° C. was added 10.05 g of Brij 35 and the solution was then allowed to cool to 55° C. To this solution 0.2007 g of EDTA dihydrate, 2.31 g of glycerin, 0.1892 g of boric acid and 0.4412 g sorbic acid were added and stirred until dissolved. The solution was held at a temperature of 55° C. for 30 minutes. The solution was allowed to cool to room temperature. This solution was designated Phase ID.

To 0.7985 g of ethanol was added 0.2012 g of cyclosporin with mixing until the cyclosporin completely dissolved. Polysorbate 80 (1.0742 g) was then added to the solution and stirred until dissolved. This solution was designated Phase IID.

The Phase IID solution was quantitatively added to the Phase ID solution. Overnight mixing completely dissolved the cyclosporin. The solution was diluted to a final weight of 200.02 g and designated Sample D.

### Sample E

To 160.81 g of water heated to 70° C. was added 14.01 g of Brij 35 and the solution was allowed to cool to 55° C. To this solution 0.1997 g of EDTA dihydrate, 2.32 g of glycerin, 0.1917 g of boric acid and 0.4405 g sorbic acid were added and stirred until dissolved. The solution was held at a temperature of 55° C. for 30 minutes and then was allowed to cool to room temperature. This solution was designated Phase IE.

To 0.7982 g of ethanol was added 0.4024 g of cyclosporin with mixing until the cyclosporin completely dissolved. Polysorbate 80 (1.0765 g) was then added to the solution and stirred until dissolved. This solution was designated Phase IIE.

The Phase IIE solution was quantitatively added to Phase IE solution. Overnight mixing completely dissolved the cyclosporin. The solution was diluted to a final weight of 200.03 g and designated Sample E.

### Sample F

To 163.99 g of water heated to 70° C. was added 13.99 g of Brij 35 and the solution was then allowed to cool to 55° C. To this solution 0.0695 g of norketotifen fumarate, 0.2009 g of EDTA dihydrate, 2.39 g of glycerin, 0.1904 g of boric acid and 0.4403 g sorbic acid were added and stirred until dissolved. The solution was held at a temperature of 55° C. for 30 minutes and then allowed to cool to room temperature. This solution was designated Phase IF.

To 0.7935 g of ethanol was added 0.2001 g of cyclosporin with mixing until the cyclosporin completely dissolved. Polysorbate 80 (1.0769 g) was added to the solution and stirred until dissolved. This solution was designated Phase IIF.

The Phase IIF solution was quantitatively added to the Phase IF solution. Overnight mixing completely dissolved the cyclosporin. The solution was diluted to a final weight of 200.03 g and designated Sample F.

TABLE 1
Representative Formulations (amounts are in % w/v)

| | Control | Sample | Sample | Sample | Sample |
|---|---|---|---|---|---|
| Component | % | A % | B % | C % | D % |
| cyclosporin | 0.1 | 0.1 | 0.1 | 0.1 | 0.1 |
| Polyoxy 40 stearate | 7.0 | 7.0 | | | |
| Polyoxyethylene | | | 7.0 | 6.0 | 5.0 |
| alkyl ether (Brij 35) | | | | | |
| EDTA | 0.1 | 0.1 | 0.1 | 0.1 | 0.1 |
| NaCl | .3 | | | | |
| glycerin | | 1.15 | 1.15 | 1.15 | 1.15 |
| boric acid | 0.095 | 0.095 | 0.095 | 0.095 | 0.095 |
| sorbic acid | 0.22 | 0.22 | 0.22 | 0.22 | 0.22 |
| sodium | 0.04 | | | | |
| metabisulfite | | | | | |
| ethanol USP | 0.395 | 0.395 | 0.395 | 0.395 | 0.395 |
| Polysorbate 80 | 0.537 | 0.537 | 0.538 | 0.537 | 0.538 |
| pH | 7.2 | 5.5 | 5.5 | 5.5 | 5.5 |

TABLE 2
Representative Formulations of cyclosporin and
cyclosporin combinations (amounts are in % w/v)

| Component | Sample E % | Sample F % |
|---|---|---|
| cyclosporin, USP: | 0.20 | 0.10 |
| norketotifen fumarate, USP | | 0.0345 |
| Brij 35, USP | 7.00 | 7.00 |
| EDTA dihydrate, USP | 0.10 | 0.10 |
| glycerin, USP | 1.15 | 1.15 |
| boric acid, NF | 0.095 | 0.095 |
| sorbic acid, NE | 0.270 | 0.220 |
| ethanol USP | 0.395 | 0.395 |
| Polysorbate 80, NF | 0.537 | 0.537 |
| pH | 5.5 | 5.5 |

Formulations comprising cyclosporin free or substantially free of sodium chloride and sodium metabisulfite were prepared (Samples A, B, E and F) with adjusted initial pH values of about 5.5. Controls comprising cyclosporin with sodium chloride and sodium metabisulfite (Control) were also prepared. The formulations and the control samples were tested for their stability at various temperatures and relative humidities (RHs). Degradation analysis of the active ingredients in the formulations was performed using HPLC using control samples for cyclosporin and norketotifen. Stability data of the compositions are summarized in Table 3. The data of Table 3 shows that compositions substantially free of sodium metabisulfite have comparable stability compared to control samples that contain sodium metabisulfite.

TABLE 3
Stability data of cyclosporin compositions.

| | Storage | Initial | Week 1 | Week 2 | Week 3 | Week 4 |
|---|---|---|---|---|---|---|
| Sample | Condition | (mg/mL) | (mg/mL) | (mg/mL) | (mg/mL) | (mg/mL) |
| Control | 5° C. | 1.0197 | 0.9860 | 0.9807 | 0.9560 | 0.9659 |
| (BCL331-134-2) | 25° C./40% RH | N/A | 0.9835 | 0.9574 | 0.9672 | 0.9713 |
| pH = 7.19 | 40° C./75% RH | N/A | 0.9738 | 0.9448 | 0.9627 | 0.9688 |
| Osmolality = 288 | 55° C. | N/A | 1.0591 | 0.9284 | 0.9351 | 0.9581 |
| mOsm/kg | | | | | | |
| A | 5° C. | 1.0496 | 1.0094 | 0.9521 | 0.9945 | 0.9799 |
| (BCL331-134-3) | 25° C./40% RH | N/A | 1.0279 | 1.0009 | 0.9896 | 0.9335 |
| pH = 5.49 | 40° C./75% RH | N/A | 1.0037 | 0.9834 | 1.0483 | 0.9901 |
| Osmolality = 317 | 55° C. | N/A | 1.0083 | 0.9647 | 0.9611 | 0.9647 |
| mOsm/kg | | | | | | |
| B | 5° C. | 1.0259 | 0.9917 | 0.9582 | 0.9552 | 0.9729 |
| (BCL331-134-4) | 25° C./40% RH | N/A | 0.9985 | 0.9566 | 0.9249 | 0.9590 |
| pH = 5.48 | 40° C./75% RH | N/A | 0.9903 | 0.9599 | 0.9040 | 0.9567 |
| Osmolality = 285 | 55° C. | N/A | 0.9846 | 0.9523 | 0.8961 | 0.9367 |
| mOsm/kg | | | | | | |
| E | 5° C. | 1.9795 | 1.9519 | 1.7698 | 1.7160 | 1.8412 |
| (BCL331-134-7) | 25° C./40% RH | N/A | 1.8279 | 1.7689 | 1.7741 | 1.8150 |
| pH = 5.52 | 40° C./75% RH | N/A | 1.8734 | 1.7500 | 1.8072 | 1.7911 |
| Osmolality = 324 | 55° C. | N/A | 1.8582 | 1.7820 | 1.7232 | 1.7590 |
| mOsm/kg | | | | | | |
| F | 5° C. | 1.0133 | 0.9762 | 0.9214 | 0.9478 | 0.9371 |
| (BCL331-134-8) | 25° C./40% RH | N/A | 0.9750 | 0.9450 | 0.9480 | 0.9322 |
| pH = 5.51 | 40° C./75% RH | N/A | 0.9686 | 0.9266 | 0.9416 | 0.9269 |
| Osmolality = 361 | 55° C. | N/A | 0.9732 | 0.9418 | 0.9076 | 0.9294 |
| mOsm/kg | | | | | | |
| F* | 5° C. | 0.3482 | 0.3478 | 0.3340 | 0.3293 | 0.3366 |
| (BCL331-134-8) | 25° C./40% RH | N/A | 0.3429 | 0.3239 | 0.3147 | 0.3199 |
| pH = 5.51 | 40° C./75% RH | N/A | 0.3309 | 0.3022 | 0.2725 | 0.2774 |
| Osmolality = 361 | 55° C. | N/A | 0.3073 | 0.2592 | 0.2105 | 0.2064 |
| mOsm/kg | | | | | | |

*Stability data of norketotifen in cyclosporin/norketotifen composition.

### Example 2

Samples having the compositions and pH values shown in Table 4 were prepared. The samples were tested for stability after having been stored at 25° C. for three months. As can be seen, the sorbic acid concentration was higher and thus sorbic acid was more stable in compositions having a pH of about 6.0 to 6.5.

TABLE 4
Stability data of cyclosporin compositions

| | | Sorbic Acid Concentration (%) | | |
|---|---|---|---|---|
| Component | Initial % | After 3 months storage at 25 C. | | |
| Cyclosporin | 0.100 | | | |
| Polyoxy 40 stearate | 7.000 | | | |
| EDTA | 0.100 | | | |
| Glycerin | 1.150 | | | |
| Boric Acid | 0.095 | | | |
| Sorbic Acid | 0.220 | 0.13 | 0.21 | 0.21 |
| Polysorbate 80 | 0.537 | | | |
| Ethyl Alcohol | 0.395 | | | |
| | | pH = 5.5 | pH = 6.0 | pH = 6.5 |

While the invention has been described in detail and with reference to specific embodiments thereof, it will be apparent to one skilled in the art that various changes and modifications can be made without departing from the spirit and scope of the invention.

We claim:

1. An aqueous ophthalmic composition comprising:
(a) a cyclosporin in an amount of from about 0.001% to about 1%;
(b) glycerin in an amount between about 0.1% and about 5%; and
(c) purified water;
(d) wherein the composition contains less than about 0.3% sodium chloride and less than about 0.04% sodium metabisulfite.

2. The aqueous ophthalmic composition of claim 1, wherein the pH of the aqueous ophthalmic composition is between about 6.0 and about 7.5.

3. The aqueous ophthalmic composition of claim 1, wherein the pH of the aqueous ophthalmic composition is about 6.5.

4. The aqueous ophthalmic composition of claim 1, wherein the composition is substantially free of sodium chloride.

5. The aqueous ophthalmic composition of claim 1, wherein the composition is substantially free of sodium metabisulfite.

6. The aqueous ophthalmic composition of claim 1, wherein the composition is substantially free of sodium chloride and sodium metabisulfite and is stable.

7. The aqueous ophthalmic composition of claim 1, further comprising a polyoxyethylene sorbitan fatty acid ester and a polyoxyethylene fatty acid ester in a total amount between about 7% and about 8%.

8. The aqueous ophthalmic composition of claim 7, wherein the polyoxyethylene sorbitan fatty acid ester and the polyoxyethylene fatty acid ester each have a HLB number between about 15 to about 17.

9. The aqueous ophthalmic composition of claim 7, wherein the polyoxyethylene sorbitan fatty acid ester is present in an amount between about 0.50% and about 0.55% and the polyoxyethylene fatty acid ester is present in an amount of about 7%.

10. The aqueous ophthalmic composition of claim 7, wherein the polyoxyethylene sorbitan fatty acid ester is polyoxyethylene 20 sorbitan monooleate.

11. The aqueous ophthalmic composition of claim 7, wherein the polyoxyethylene fatty acid ester is polyoxyethylene 40 monostearate.

12. The aqueous ophthalmic composition of claim 1, further comprising ethanol in an amount from about 0.2% to about 0.5%.

13. The aqueous ophthalmic composition of claim 1, further comprising boric acid in an amount from about 0.01% to about 0.2%.

14. The aqueous ophthalmic composition of claim 1, further comprising sorbic acid in an amount from about 0.01 to about 0.5%.

15. The aqueous ophthalmic composition of claim 14, wherein the sorbic acid is in an amount from about 0.2 to about 0.3% and the composition is stable.

16. The aqueous ophthalmic composition of claim 1, further comprising ethylene diamine tetraacetic acid in an amount from about 0.01% to about 1%.

17. The aqueous ophthalmic composition of claim 1, further comprising a therapeutically effective amount of a member selected from the group consisting of an antihistamine, a mast cell stabilizer, a steroidal anti-inflammatory agent, a non-steroidal anti-inflammatory agent, and mixtures thereof.

18. A method of treating an ocular condition comprising contacting ocular tissue with an aqueous composition comprising:
(a) a cyclosporin in an amount of from about 0.001% to about 1%;
(b) glycerin in an amount between about 0.1% to about 5%; and
(c) purified water;
(d) wherein the composition contains less than about 0.3% sodium chloride and less than about 0.04% sodium metabisulfite.

19. The method of claim 18, wherein the composition further comprises sorbic acid in an amount from about 0.01 to about 0.5% and the composition is stable.

20. An aqueous ophthalmic composition comprising:
(a) a cyclosporin in an amount of 0.1%;
(b) glycerin in an amount of 1.15%;
(c) polyoxy 40 stearate in an amount of 7%;
(d) EDTA in an amount of 0.1%;
(e) boric acid in an amount of 0.095%;
(f) sorbic acid in an amount of 0.22%;
(g) polysorbate 80 in an amount of 0.537%; and
(h) ethyl alcohol in an amount of 0.395%.
(i) wherein the composition contains less than about 0.3% sodium chloride and less than about 0.04% sodium metabisulfite.
